(19) United States
(12) Patent Application Publication
(10) Pub. No.: US 2010/0106016 A1
(43) Pub. Date: Apr. 29, 2010

(54) Non-Invasive Blood Pressure Monitoring Device and Method

(75) Inventors: Jorge L. Orbay, Miami, FL (US); William Garcia de Quevedo, Miami, FL (US); Alex Espinosa, Miami, FL (US); Mickey Moore, Miami, FL (US)

Correspondence Address: LERNER GREENBERG STEMER LLP, P O BOX 2480, HOLLYWOOD, FL, 33022-2480, US

(73) Assignee: Skeletal Dynamics LLC, Miami, FL (US)

(21) Appl. No.: 12/604,995

(22) Filed: Oct. 23, 2009

Related U.S. Application Data
(60) Provisional application No. 61/107,891, filed on Oct. 23, 2008.

(51) Int. Cl. A61B 8/04; A61B 5/025

(52) U.S. Cl. 600/437; 600/494

(57) ABSTRACT

A device and method for measuring blood pressure are provided. More particularly, a non-invasive device utilizing an ultra-sound transducer and a conventional blood pressure cuff are used, in combination, to measure blood pressure.

### CROSS-REFERENCE TO RELATED APPLICATIONS

The present application claims priority to co-pending Provisional Patent Application No. 61/107,891, filed on Oct. 23, 2008 and entitled “Non-Invasive Blood Pressure Monitoring Device And Method”; that application being incorporated herein, by reference, in its entirety.

### BACKGROUND OF THE INVENTION

a. Field of the Invention

The present invention relates to a device and method for measuring blood pressure, and more particularly, to a non-invasive device and method for measuring blood pressure utilizing an ultra-sound transducer and a conventional blood pressure cuff.

b. Description of the Related Art

Blood Pressure Monitoring is essential in the care of patients during surgery and in the ICU setting. To date there is no reliable method of instantaneously measuring blood pressure in a non-invasive way. The usual method used for non-invasive blood pressure measurement is to use a blood pressure cuff. This is a device consisting of an inflatable cuff connected to an air pump and a pressure transducer. The cuff is applied around a limb, usually the upper arm, and inflated to a pressure above the systolic (highest) pressure; the cuff is slowly deflated and the pressure at which blood first starts to pass through the artery underneath the cuff is recorded. The signal used to ascertain that the cuff has reached systolic pressure is the sound produced by the blood flowing through the underlying artery. This sound is pulsatile and either heard by the examiner via a stethoscope or detected by a machine that performs the operation automatically. When the cuff pressure is between systolic and diastolic (lowest pressure), the blood will flow in an intermittent manner through the artery underneath the cuff and produce a characteristic sound. When the pressure in the cuff reaches diastolic pressure, blood flow will become continuous and the sound will disappear, this signals the examiner that diastolic pressure has been reached. This operation is performed automatically by a machine every 3 minutes in the operating room and can also be performed by the touch of a button at the anesthesiologist's need. The problem with this method is that blood pressure may reach dangerous levels for a significant period of time before there is any evidence that such an event is occurring. Instantaneous and continuous measurement of blood pressure (beat to beat blood pressure monitoring) is available today through an arterial line. This method uses an indwelling catheter placed inside the lumen of an artery which is physically connected to a pressure transducer. Arterial lines are effective but invasive and can lead to serious damage to the tissues downstream to the catheter therefore; they are only used in very special situations such as open heart surgery. Non invasive methods of estimating blood pressure by correlating cuff measurements to mechanical sensing of the actual pulsation of peripheral arteries (usually the radial) by means of stress sensors have been attempted and commercialized but have proven unreliable. The reason for this is the variable thickness of the tissues overlying the small peripheral arteries that are accessible to this method and the fact that the pressure in small peripheral arteries frequently does not correlate well to central arterial pressure.

### DESCRIPTION OF THE PREFERRED EMBODIMENTS

This invention provides non-invasive continuous (beat-to-beat) measurement of arterial pressure by combining conventional blood pressure cuff measurements, an imaging device, such as ultrasound, to monitor the size, shape and behavior of the underlying artery and a digital processor to create a virtual mechanical model of the artery. Instantaneous beat-to-beat blood pressure is calculated by correlating the model with the anatomical information obtained from the imaging device.

The invention comprises a blood pressure cuff that is connected to or incorporates an ultrasound transducer/transceiver and is applied around a limb and over a large artery such as the brachial artery at the upper arm. The ultrasound transceiver can be located proximal to the blood pressure cuff, but, in one preferred embodiments, is incorporated into the blood pressure cuff, in communication with the blood pressure cuff. Alternately, in another preferred embodiment, the ultrasound transducer can be removably affixed to the blood pressure cuff, for example, using a hook and loop type fastener, such as is sold under the brand name VELCRO™ Alternately, and less preferably, the ultrasound transceiver can be affixed to the skin of the patient, using an adhesive and/or tape. However, it can be recognized that certain advantages, such as ease of use, are provided by providing the blood pressure cuff and ultrasound transceiver in a single unit (i.e., integrated and/or previously connected together).

In the instant invention, the ultrasound transceiver of the blood pressure cuff/ultrasound device generates ultrasound waves that travel into the arm and bounce back preferentially from fluid filled structures such as arteries and veins. The signal that returns to the ultrasound transceiver is captured and relayed to a processor, which interprets the information by means of dedicated circuitry. Such information can be relayed to the processor either wirelessly, using the appropriate transmission electronics, or by wired communication. Using the Doppler Effect, the processor determines which vessel is the artery; this is possible due to differences in the velocity and waveform of the flow. The processor then measures and correlates vessel parameters such as cross-sectional area of the artery with blood pressure measurements, as determined by the cuff, to digitally calculate the vessel's mechanical properties, such as compliance, and to create a digital model of the vessel. This model is used by the processor to calculate instantaneous blood pressure based on the anatomical information provided by the transceiver in the intervals between cuff measurements. In other words, the vessel itself is used as a pressure transducer, once its properties have been ascertained. An arterial pressure wave is caused by the pumping action of the heart and, therefore, the measured vessel parameters must be properly timed into this cycle. In one particular embodiment of the instant invention, the timing of the cycle can be achieved by connecting the device's processor an electrocardiogram lead which would signal the ultrasound transceiver when to capture an image.

During each cycle, the cross-sectional area of the detected artery can be measured at the peak of arterial pressure and a second cross-sectional measurement can be obtained at the trough. Comparing these two with systolic and diastolic pressures will yield the vessel's compliance. Also the instantaneous blood pressure value can be obtained from equation (1), as follows:

P=2πL_{I}(1−r₀/r) (1)

### Where:

P=Pressure;

L_{I}=Vessel Coefficient of elasticity;

r₀=Resting vessel radius; and

r=Instantaneous vessel radius.

Velocities of wall expansion as well as wall acceleration are parameters that may also be used to augment the virtual model of vessel behavior. All these measurements are repeated continuously in order to constantly recalibrate the instrument during the period of use.

The above-described device of the present invention can additionally be used in an inventive method to non-invasively determine the blood pressure of a patient under emergency conditions, such as during surgery, or in while the patient is in an intensive care unit (ICU). For example, the device of the particular invention can be applied to the arm of a patient, with the ultrasound transceiver being located over the patient's brachial artery. In a preferred embodiment of the present invention, wherein the ultrasound transceiver is removably connected to and/or integrated with a blood pressure cuff, the ultrasound transceiver is located proximal to the desired artery by affixing the blood pressure cuff to the upper arm of the patient.

In one particular embodiment of the present invention, ultrasound readings are taken at a single location along the artery to determine, among other characteristics, the cross-sectional area of the artery at the peak and trough of a cycle. These instantaneous cross-sectional areas of the artery are used to form a rough correlation to instantaneous blood pressure of a patient. For example, a memory device in communication with the processor can store a look-up table correlating each discrete cross-sectional area of the artery to, roughly, an associated, blood pressure. Alternately, known equations can be used to convert the detected cross-sectional areas of the artery to an associated blood pressure. Using such look-up table or equations, the processor is able to determine a surge or pulse that would correlate to an unacceptably high instantaneous blood pressure in the patient. For example, the processor determines whether the rough, instantaneous blood pressure of the patient exceeds a threshold value set by the user and/or by the system software. In response to a determination by the processor that the cross-sectional area(s) of the artery correlates, roughly, to an unacceptably high blood pressure of the patient, the system will trigger the operation and inflation of the blood pressure cuff, in order to obtain a more accurate blood pressure reading for the patient. If the blood pressure for the patient measured by the blood pressure cuff additionally indicates an unacceptably high blood pressure of the patient (i.e., exceeding a preset threshold), an alarm is triggered. Such alarm can be provided locally to the patient, on electrical monitors and biometric readout displays (i.e., in the operating room or ICU), and remotely, for example, at a remote nurses' station and/or doctor's area. Such alarm informs the patient's caretaker of a change in the patient's status so that corrective action can be taken.

Additionally, in one preferred embodiment of the present invention, software in communication with the processor can determine the compliance of the measured artery, as discussed above. Such software can be used to determine how “sick” is the selected artery. Using the information regarding the condition and elasticity of the artery, the system can be adjusted to each individual patient.

For example, depending on the elasticity of the artery, a user of the system, or the software itself, could set and/or adjust the parameters necessary for triggering the operation of the blood pressure cuff. Additionally, in one particular embodiment, using the information regarding the elasticity or “sickness” of the measured artery, the system could adjust what values of cross-sectional area of the artery correspond to which blood pressures in this particular patient. Then, the system could trigger the operation of the blood pressure cuff, and subsequently, the alarm, when the cross-sectional area of the artery of the particular patient correlates to an unacceptably high blood pressure, wherein such determination takes into account the actual characteristics of each patient's artery. For example, in one particular embodiment of the system of the instant invention, the system can be programmed to trigger the operation of the blood pressure cuff when the cross-sectional area of the artery of the particular patient (i.e., factoring the elasticity of this patient's artery) correlates to a blood pressure of 200 systolic/120 diastolic, or higher. Alternately, the amount of change in cross-sectional area from the peak measurement of the trough measurement of the cycle, adjusted for the individual characteristics of each patient's artery, can be used to trigger the operation of the blood pressure cuff.

In one particular embodiment of the instant invention, in a patient having ideal artery characteristics, a pulse or surge that results in a change of cross-sectional measurement of 30% or more, between two cycles of peak measurements and/or trough measurements, could trigger the operation of the blood pressure cuff and, resultantly, of an alarm. However, other factors and/or amounts of change can be pre-programmed into the system, for triggering the operation of the blood pressure cuff.

In summary, the instant invention includes a combined ultrasound transceiver/blood pressure cuff device and system for roughly determining, through ultrasound information obtained at a particular (i.e., single) location along the patient's artery, significant changes in the arterial cross-section, and correspondingly, in the blood pressure of a patient. Upon determination of a significant change, a more accurate blood pressure reading can be taken with the blood pressure cuff. Confirmation of an unacceptably high blood pressure reading from the blood pressure cuff can be, resultantly signaled to the patient's caregiver. The determination of what amount constitutes a “significant change” can be determined for each patient using characteristics of that patient's artery, as obtained from the received ultrasound information. As such, the trigger points of the system can be tailored to the personal characteristics of each individual patient.

We claim:

1. A system for monitoring the blood pressure of a patient, comprising:
a blood pressure cuff located around the arm of the patient;
an ultrasound transceiver located in communication with the blood pressure cuff;
a processor for receiving and processing information from the ultrasound transceiver, said processor triggering the operation of said blood pressure cuff when information received from the ultrasound transceiver indicates a blood pressure of the patient exceeds a preset threshold value.

2. The system of claim 1, wherein the ultrasound transceiver is located on the blood pressure cuff.

3. The system of claim 1, wherein the ultrasound transceiver is removably connected to the blood pressure cuff.

4. The system of claim 1, wherein said processor uses the ultrasound information to determine at least one characteristic of the patient's artery and wherein said threshold is adjusted based on said at least one characteristic.

5. The system of claim 4, wherein said threshold is manually adjusted.

6. The system of claim 4, wherein said threshold is automatically adjusted by said processor.

7. A method for monitoring the blood pressure of a patient, comprising the steps of:
providing a blood pressure cuff located around the arm of the patient;
providing an ultrasound transceiver located in communication with the blood pressure cuff;
processing information from the ultrasound transceiver; and
triggering the operation of the blood pressure cuff when information received from the ultrasound transceiver indicates a blood pressure of the patient exceeds a preset threshold value.

8. The method of claim 7, further comprising the step of triggering an alarm if the blood pressure measured by the blood pressure cuff exceeds a second preset threshold.

9. The method of claim 7, further comprising the steps of:
determining, from the ultrasound information, at least one characteristic of the patient's artery; and
adjusting the threshold based on the at least one characteristic.
